(19) United States
(12) Patent Application Publication
(10) Pub. No.: US 2010/0028430 A1
(43) Pub. Date: Feb. 4, 2010

(54) MATERIAL AND MANUFACTURING METHOD OF BIOACTIVE PROTEIN-CALCIUM PHOSPHATE COMPOSITE

(75) Inventors: Ju Woong Jang, Seoul (KR); Kwang II Lee, Gyeonggi-do (KR); Hyang Kim, Gyeonggi-do (KR); Young Bock Shim, Seoul (KR); Hyun Min Kim, Gyeonggi-dol (KR)

Correspondence Address: MERCHANT & GOULD PC, P.O. BOX 2903, MINNEAPOLIS, MN, 55402-0903, US

(73) Assignee: Korea Bone Bank Co., Ltd., Seocho-ku (KR)

(21) Appl. No.: 12/063,195

(22) PCT Filed: Jul. 22, 2006
(86) PCT No.: PCT/KR2006/002899

§ 371 Date: Jul. 16, 2009

(30) Foreign Application Priority Data
Jun. 13, 2006 (KR) 10-2006-0052960

(51) Int. Cl. A61K 38/16; A61K 9/14; A61P 19/08; C12N 5/071

(52) U.S. Cl. 424/484; 514/12; 435/325

(57) ABSTRACT

Disclosed are a bioactive protein-calcium phosphate ceramic composite for surface modification of a substrate for use as a substitute in the treatment of musculoskeletal disorders, and a preparation method thereof. The bioactive protein-calcium phosphate ceramic composite is prepared by mixing an aqueous solution of calcium phosphate and a protein, and impregnating the calcium phosphate and the protein in the resulting aqueous solution to co-precipitate them on a substrate, such as metals, ceramics and polymers, wherein the substrate is patterned, and different proteins are impregnated in at least two regions of the patterned regions.

### TECHNICAL FIELD

The present invention relates to a bioactive protein-calcium phosphate ceramic composite for surface modification of a substrate for use as a substitute in the treatment of musculoskeletal disorders, and a preparation method thereof. More particularly, the a bioactive protein-calcium phosphate ceramic composite, which is prepared by mixing an aqueous solution of calcium phosphate and a protein, and impregnating the calcium phosphate and the protein in the resulting aqueous solution to co-precipitate them on a substrate, wherein the bioactive protein-calcium phosphate ceramic composite is patterned on the surface of the substrate, and different proteins impregnate at least two regions of the patterned regions, and a preparation method thereof.

### BACKGROUND ART

Generally, the treatment of damage to organs, tissues or bones in humans incurs high expenses. Such high expenses are a severe problem in modern society, which has not sufficiently secured medical care.

Damaged organs, tissues or bones have been treated by transplanting organs, tissues or bones from donors to patients, or by transplanting artificially prepared organs or bones to patients.

Recent studies have involved not implants in inactive states, in which the implants do not interact with biological tissues, but implants having biological activity in biological tissues. To obtain such implants having biological activity, material having good surface properties and being finely designed, such as ceramics, metals and polymers, should be used. These materials actively react with surrounding biological tissues, thereby allowing implants to replace biological tissues.

For example, a technique of preparing an artificial hip joint is based on applying hydroxyapatite to a base material. A technique of preparing artificial blood vessels is based on applying algin and collagen to a base material.

More recently, some studies have focused on the goal of engineering tissue on complex-type artificial biological organs, which employ both cells extracted from biological tissues and a base material. Such artificial organs are considered to be different from the previous concept of complete replacement of biological tissues by artificial organs, and thus as the restoration of damaged tissues to original states.

The complex-type artificial biological organs, in which cells or proteins having specific functions in organs are attached to a base material and cultivated therein, enable the regeneration of tissues or organs for which restoration is desired.

For example, Japanese Pat. Laid-open Publication No. 2003-187396 discloses titanium or a titanium alloy on which a protein, such as a growth factor or a cell adhesion factor, is supported. The protein-supported titanium or titanium alloy should be biocompatible, and can be used as a biological tissue replacement material stimulating the reconstruction of biological tissues, an artificial bone, an artificial tooth root, an anti-coagulating material, and a support for tissue engineering.

The published invention employs biologically active substances, such as growth factors, cell adhesion factors, other proteins, phospholipids, polysaccharides, and hormones, in order to achieve biological tissue reconstruction, tissue induction and cellular differentiation. The published invention states that in this case, since mechanical intensity above a certain level is required, titanium or titanium alloy should be mainly used. The published invention also states that in order to prepare artificial heart and artificial tooth roots, biologically active substances must be supported on titanium or titanium alloy, thereby supporting cells thereon or achieving tissue reconstruction.

In addition, the published invention emphasizes that since the co-precipitation of calcium phosphate and a protein on a simple titanium metal surface results in small amounts of the protein being supported, the titanium surface should be treated with alkali to increase the amount of protein supported.

However, the above invention does not provide the localization of tissue growth because a single base material (titanium) allows only a single tissue to grow during tissue reconstruction. Thus, it is not easy to organize several different growth factors through a single reconstruction and to transplant them to a desired area of the body.

Moreover, the above invention employs expensive titanium as a base material, neglecting the need to reduce the enormous expense upon implantation. Thus, there is a need for diversification of the base material.

### DISCLOSURE

### Technical Solution

Accordingly, the present invention has been made keeping in mind the above problems occurring in the prior art, and an object of the present invention is to provide a bioactive protein-calcium phosphate ceramic composite promoting the reconstruction of biological tissues and a method of preparing the composite, the composite exhibiting the innate physiological activity of the protein at ambient temperature and under ambient pressure and regenerating all tissues in the musculoskeletal system regardless of the type, structure and shape of a base material.

In addition, the present invention aims to produce various tissue cultures that are locally activated and to produce a composite tissue in which blood vessels and other tissues coexist by patterning a coating of the composite tissue on the surface of a substrate and locally selecting tissues on the patterned substrate to locally culture tissues only on the selected position.

### DESCRIPTION OF DRAWINGS

FIG. 1 schematically illustrates the formation of a nanocomposite of a physiologically active protein and a bioactive calcium phosphate ceramic and the formation of a coating of the composite on the surface of a substrate according to the present invention;

FIG. 2 schematically illustrates standard, control and test groups for the formation of a nanocomposite of a physiologically active protein and a bioactive calcium phosphate ceramic according to the present invention;

FIG. 3 shows scanning electron microscopy (SEM) images of the surface of a substrate measuring 5 mm>4 mm (1 mm thick), which was impregnated with 5 ml of physiological saline, a calcium phosphate (CaP) solution, and CaP solution plus 10 μg/ml recombinant human BMP2, on which a recombinant human BMP2-CaP nanocomposite was deposited;

FIG. 4 shows TF-XRD patterns of a substrate measuring 7 mm>7 mm (1 mm thick), which was impregnated with 18 ml of physiological saline, a CaP solution, and CaP solution plus 10 μg/ml recombinant human BMP2, on which a recombinant human BMP2-CaP nanocomposite was deposited;

FIG. 5 shows an FT-IRRS spectrum of the recombinant human BMP2-CaP nanocomposite of FIG. 4;

FIG. 6 shows the distribution of a BMP2-calcium phosphate nanocomposite on the surface of a substrate measuring 5 mm>4 mm (0.3 mm thick), which was impregnated with 5 ml of a calcium phosphate (CaP) solution, CaP solution plus 1 μg/ml recombinant human BMP2, and CaP solution plus 10 μg/ml recombinant human BMP2, on which a recombinant human BMP2-CaP nanocomposite was deposited, and which was incubated in an anti-human BMP2 antibody;

FIG. 7 shows the expression levels of osteogenic marker genes on a BMP2-CaP nanocomposite-deposited substrate to which MC3T3-E1 cells were attached, wherein gene expression levels were analyzed using RT-PCR, and the optical density of PCR bands was determined; and

FIG. 8 shows SEM images for the adhesion and differentiation of mouse MC3T3-E1 osteoblastic cells over a recombinant human BMP2-CaP nanocomposite-deposited substrate, wherein cells were seeded on the substrate at a density of 2×10⁴ cells per 5 mm>4 mm of substrate, and were allowed to grow for three days.

### BEST MODE

In order to achieve the above objects, the present invention provides a bioactive protein-calcium phosphate ceramic composite, which is prepared by mixing an aqueous solution of calcium phosphate and a protein, and impregnating the calcium phosphate and the protein in the resulting aqueous solution to co-precipitate them on a substrate, wherein a coating of the composite is patterned, and different proteins are impregnated on at least two regions of the patterned regions.

The aqueous solution of calcium phosphate preferably contains 130-160 mM NaCl, 1-3 mM K₂HPO₄.3H₂O, and 2-5 mM CaCl₂, and is adjusted to a pH of 7-8. The protein is preferably at least one selected from among bone morphogenetic protein (BMP), VGF, TGF, and DBM.

A mixture of 20˜40 ml of the aqueous solution of calcium phosphate and 0.1˜100 g/ml of the protein such as BMP is preferably co-precipitated on a substrate.

The protein layer is preferably 0.1-1000 μm thick.

The protein-calcium phosphate ceramic composite is preferably matured at 20-30° C. for 1-5 days after being impregnated.

The calcium phosphate ceramic preferably contains calcium and phosphorus within a Ca/P atomic ratio of 1.0˜2.0, which is similar to that of inorganic matter in hard tissues of the body.

The present invention also provides a method of preparing a bioactive protein-calcium phosphate ceramic composite, comprising forming a pattern on a substrate, co-precipitating a mixture of an aqueous solution of calcium phosphate and a protein on at least one region of exposed regions of the substrate, and growing a biological tissue on the co-precipitated substrate. Preferably, the method further includes co-precipitating the mixture of the aqueous solution of calcium phosphate and the protein on at least one other region in addition to the co-precipitated region.

The aqueous solution of calcium phosphate preferably contains 130˜160 mM NaCl, 1˜3 mM K₂HPO₄3H₂O, and 2˜5 mM CaCl₂, and is adjusted to pH 7˜8.

The protein is preferably at least one selected from among bone morphogenetic protein (BMP), VGF, TGF, and DBM.

A mixture of 20˜40 ml of the aqueous solution of calcium phosphate and 0.1˜100 g/ml of the protein such as BMP is preferably co-precipitated on a target substrate, such as metal, polymer, or ceramic.

The protein layer is preferably 0.1˜1000 μm thick.

The protein-calcium phosphate ceramic composite is preferably matured at 20˜30° C. for 1˜5 days after being impregnated.

The calcium phosphate ceramic preferably contains calcium and phosphorus within a Ca/P atomic ratio of 1.0˜2.0.

A better understanding of the present invention may be obtained with reference to the accompanying drawings and through the following examples which are set forth to illustrate, but are not to be construed as the limit of the present invention.

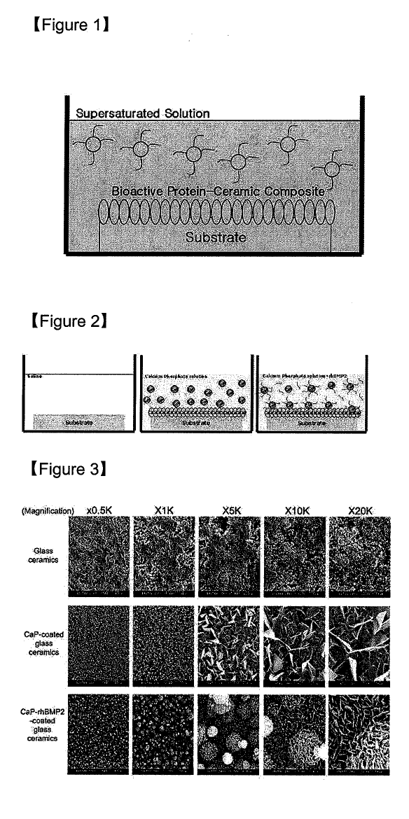

FIG. 1 schematically represents the formation of a nanocomposite of a physiologically active protein and a bioactive calcium phosphate ceramic and the formation of a coating of the composite on the surface of a substrate according to the present invention.

As illustrated in FIG. 1, the present invention relates to a bioactive protein-calcium phosphate ceramic composite, which is prepared by mixing an aqueous supersaturated solution of calcium phosphate and recombinant human BMP-2, and depositing the calcium phosphate and the protein on a substrate as a base material. The base material is patterned (not shown) in a manner of locally forming a protein-ceramic composite corresponding to the pattern. If desired, one or more proteins as an ingredient of the composite may be selected from among BMP, VGF, TGF, DBM, and the like, mixed, and deposited on the substrate. The present invention is characterized by providing a patterned substrate having physiologically active functions thereon.

It will be apparent to those skilled in the art that the protein is not limited to the above examples, but that numerous other proteins can be employed.

In addition, the present invention experimentally revealed that a protein-calcium phosphate ceramic nanocomposite, which is formed by mixing a ceramic, particularly calcium phosphate ceramic, with a protein, is able to grow on a substrate having specific features, and is also able to grow on a substrate regardless of the substrate type.

### MODE FOR INVENTION

A calcium phosphate ceramic coating was formed by co-precipitating bone morphogenetic protein (BMP) and calcium phosphate in a supersaturated solution of calcium phosphate to support the protein on the calcium phosphate ceramic, as follows.

### 1. Preparation of a Calcium Phosphate Solution for Protein Precipitation

A calcium phosphate solution was prepared and adjusted to pH 7.4 using NaCl (142 mM), K₂HPO₄.3H₂O (1.50 mM), CaCl₂ (3.75 mM, dissolved in ultra pure water), Buffer TRIS (50 mM) and 1 M HCl (all reagents were obtained from Nacalai Tesque Co., Japan) at 25° C.

### 2. Selection of a Substrate for Composite Coating

A variety of known and previously unknown materials, such as biocompatible metals, ceramics and polymers, can be used as a substrate for composite coating. Also, the substrate can be physically or chemically surface-treated to facilitate composite coating formation, but such surface treatment is not essential. In the present invention, the typical biocompatible crystallized glass A-W was used.

3. The Precipitation of Recombinant Human BMP2-Calcium Phosphate Composite and Surface Coating of Crystallized Glass A-W with the Composite.

FIG. 2 schematically represents standard, control and test groups for the formation of a nanocomposite of a physiologically active protein and a bioactive calcium phosphate ceramic according to the present invention. FIG. 3 shows scanning electron microscopy (SEM) images of the surface of a substrate measuring 5 mm>4 mm (1 mm thick), which was impregnated with 5 ml of physiological saline, a calcium phosphate (CaP) solution, and CaP solution plus 10 μg/ml recombinant human BMP2, wherein a recombinant human BMP2-CaP nanocomposite was deposited on the substrate.

As illustrated in FIG. 2, a substrate was cut into sections having a predetermined size for impregnation of the substrate with 30 ml of a calcium phosphate solution per 1 cm² cut area. A substrate was impregnated with physiological saline for a standard group, a calcium phosphate solution for a control group, and a mixture of a calcium phosphate solution and 0.1˜100 μg/ml recombinant human BMP2. The substrate impregnated with the solution was stored in an incubator at 25° C. for a predetermined period of time (about three days in this test) to induce deposition of a recombinant human BMP2-calcium phosphate nanocomposite on the substrate.

In order to observe the surface of the thus-deposited recombinant human BMP2-calcium phosphate nanocomposite, the surface of the substrate was analyzed using SEM, TF-XRD and FT-IRRS. In addition, in order to estimate the biological behavior of the substrate according to the recombinant human BMP2-calcium phosphate nanocomposite, the differentiation capacity of osteoblasts was assessed using mouse MC3T3-E1 osteoblastic cells.

### 3.1 SEM Imaging of the Recombinant Human BMP2-Calcium Phosphate Nanocomposite and Analysis of SEM Images

As shown in FIG. 3, a substrate measuring 5 mm>4 mm (1 mm thick) was impregnated with 5 ml of physiological saline (standard group), a calcium phosphate (CaP) solution (control group), and CaP solution plus 10 μg/ml recombinant human BMP2 (test group). The substrate impregnated with the solution was stored in an incubator at 25° C. for a period of three days to deposit a recombinant human BMP2-calcium phosphate nanocomposite thereonto. The surface of the substrate was examined with scanning electron microscopy (SEM), and SEM images were analyzed.

When the control and test groups were compared to the standard group, the surface of the control substrate was found to be uniformly coated with foliated calcium phosphate particles, and on the substrate surface of the test group, which was impregnated with CaP solution plus recombinant human BMP2, the nucleation and growth of deposits containing the protein at the center was observed.

3.2 Analysis of the Recombinant Human BMP2-Calcium Phosphate Nanocomposite using TF-XRD and FT-IRRS

FIG. 4 shows TF-XRD patterns of a substrate of 7 mm>7 mm (1 mm thick), which was impregnated with 18 ml of physiological saline, a calcium phosphate (CaP) solution, and CaP solution plus 10 μg/ml recombinant human BMP2, and was stored in an incubator at 25° C. for a period of three days to deposit a recombinant human BMP2-calcium phosphate nanocomposite thereonto. FIG. 5 shows FT-IRRS spectra of the substrate of FIG. 4.

As shown in FIGS. 4 and 5, TF-XRD and FT-IRRS revealed that a CaP ceramic coating was formed by the deposition of the substrate with a CaP solution and CaP solution plus 10 μg/ml recombinant human BMP2.

### 3.3 Immunofluorescent Staining of Recombinant Human BMP2

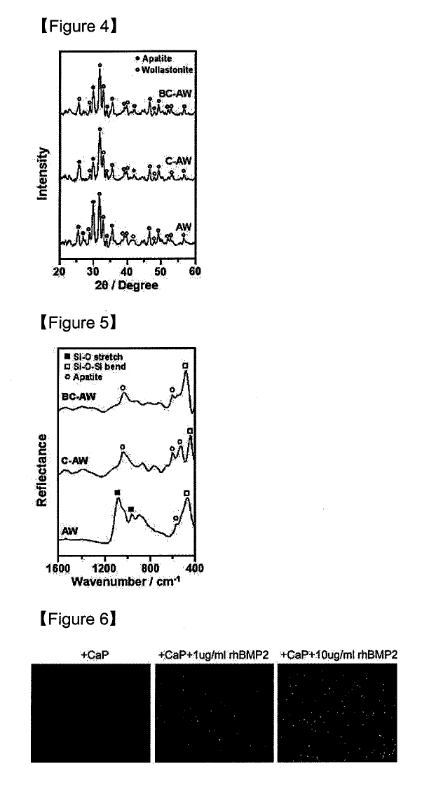

A substrate measuring 5 mm>4 mm (0.3 mm thick) was impregnated with 5 ml of a calcium phosphate (CaP) solution, CaP solution plus 1 μg/ml recombinant human BMP2, and CaP solution plus 101g/ml recombinant human BMP2, and was stored in an incubator at 25° C. for a period of three days to deposit a recombinant human BMP2-calcium phosphate nanocomposite thereonto. In order to examine the distribution of the composite on the surface of the substrate, the BMP2-calcium phosphate nanocomposite-deposited substrate was incubated in an anti-human BMP2 antibody, and was observed under a fluorescent microscope. The results are given in FIG. 6.

In detail, the surface distribution of the composite was obtained according to the following procedure.

First, a substrate was impregnated with each of the above solutions, and stored for three days. The nanocomposite-coated substrate was washed with phosphate buffered saline (PBS) two times, and fixed with 3% formaldehyde at 4° C. for 20 min. After being washed again with PBS two times or more, the substrate was incubated in a primary antibody to human BMP2 (goat polyclonal antibody, Santa Crus Biotech., Inc.), which was diluted with 1% bovine serum albumin (BSA), at room temperature for two hours.

After the substrate was washed again with PBS two times or more for 5 min each, it was incubated in a secondary antibody to human BMP2 (Fluorescent anti-goat IgG (Vector Lab., Inc.)), which was diluted with 1% BSA, at room temperature for 45 min, followed by washing with PBS two times or more for 5 min each time.

Then, a mounting medium for fluorescent microscopy was applied onto the substrate. The substrate was covered with a cover glass and analyzed for its microstructure using a confocal microscope.

As shown in FIG. 6, the deposition of the BMP2-calcium phosphate nanocomposite on the substrate increased with increasing BMP2 concentrations, and no antibody reactivity against BMP2 was observed on a substrate on which only calcium phosphate was deposited.

3.4 Induction of differentiation of osteoblasts by surface coating of a substrate with the recombinant human BMP2-calcium phosphate nanocomposite
\textcircled{1} Detection of Osteogenic Marker Gene Expression

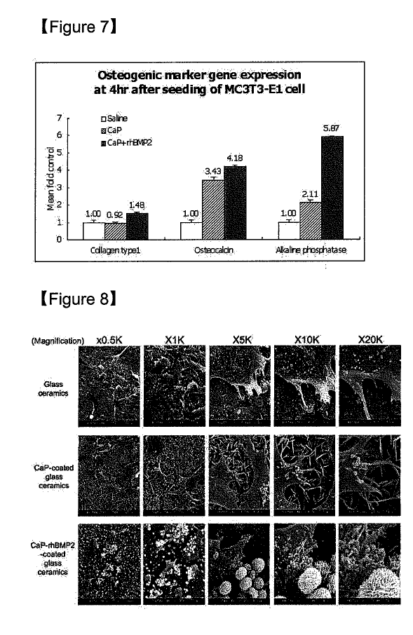

*133MC3T3-E1 cells were attached to a BMP2-CaP nanocomposite-deposited substrate, and the expression levels of osteogenic marker genes were evaluated using RT-PCR. The optical density of PCR bands was determined. The results are given in FIG. 7.

A substrate measuring 7 mm>7 mm (1 mm thick) was impregnated with 18 ml of physiological saline (standard group), a calcium phosphate (CaP) solution (control group), and CaP solution plus 10 μg/ml recombinant human BMP2 (test group), and was stored in an incubator at 25° C. for a period of three days to deposit a recombinant human BMP2-calcium phosphate nanocomposite thereonto. The substrate was subjected to TF-XRD and FT-IRRS.

Mouse MC3T3-E1 osteoblastic cells were seeded onto the recombinant human BMP2-calcium phosphate nanocomposite-deposited substrate at a density of 2×10⁴ cells per 5 mm>4 mm substrate. RNA was extracted from the cells at 4, 6, 12, 24 and 72 hrs, and analyzed using RT-PCR for the expression of osteogenic marker genes, such as collagen type I, osteocalcin, and alkaline phosphatase. PCR conditions and sequences of primers used are summarized in Table 1, below.

TABLE 1

| | | | | Product | Rxn |
|---|---|---|---|---|---|
| | | | | size | Temp. |
| Group | Primer name | | 5′ to 3′ | (bp) | (cycle) |
| 1 | Mouse | Forward | ggt aca gtg | 168 | 50(28) |
| | 18S | | aaa ctg cga | | |
| | rRNA | | at | | |
| | | Reverse | ggg ttg gtt | | |
| | | | ttg atc tga | | |
| | | | ta | | |
| 2 | Mouse | Forward | cct ggt aaa | 222 | 58(28) |
| | type (I) | | gat ggt gcc | | |
| | collagen | Reverse | cac cag gtt | | |
| | | | cac ctt tcg | | |
| | | | cac c | | |
| 3 | Mouse | Forward | cct cag tcc | 219 | 58(28) |
| | osteo- | | cca gcc cag | | |
| | calcin | | atc c | | |
| | | Reverse | cag ggc aga | | |
| | | | gag aga gga | | |
| | | | cag g | | |
| 4 | Mouse | Forward | gcc ctc tcc | 372 | 55(35) |
| | ALP | | agg aca tat | | |
| | | | a | | |
| | | Reverse | cca tga tca | | |
| | | | cgt cga tat | | |
| | | | cc | | |

The 18S rRNA gene was used as an internal control for total RNA amount. PCR products were electrophoresed on a 2% agarose gel, and the optical density of PCR bands was determined using a TINA program.

As shown in FIG. 7, 4 hrs after cell seeding, the attachment of MC3T3-E1 cells to the BMP2-CaP nanocomposite-deposited substrate (test group) resulted in an increase in expression levels of collagen type I, osteocalcin and alkaline phosphatase genes by 1.48, 4.18 and 5.87 times, respectively, compared to the standard group. The control substrate also exhibited an excellent ability to differentiate osteoblasts. In addition, 24 hrs after cell seeding, osteocalcin gene expression remarkably decreased in standard and control groups, but was maintained in the test group (BMP2-CaP nanocomposite-deposited substrate).

\textcircled{2} Analysis of Cell Morphology of MC3T3-E1 in the BMP2-CaP Nanocomposite using SEM

Mouse MC3T3-E1 osteoblastic cells were seeded on a recombinant human BMP2-CaP nanocomposite-deposited substrate at a density of 2×10⁴ cells per 5 mm>4 mm substrate, and were allowed to grow for three days. The cell adhesion and differentiation over the substrate were analyzed using SEM. The results are given in FIG. 8.

As shown in FIG. 8, no difference was observed between test and control groups in the cell adhesion, but MC3T3-E1 cells of test and control groups were healthier and more active than those of a standard group.

In particular, cells of the test group were found to be superior with respect to protein secretion onto cell surfaces and cell receptor distribution to those of the control group. Also, in the test group, cells attached to the substrate degraded BMP2-CaP nanocomposite particles coated onto the surface of the substrate and grew thereon, and an extracellular matrix for neogenesis of cells was formed around the degraded particles.

As described above, a protein-CaP composite in which two or more proteins are co-precipitated may be prepared by impregnating two or more proteins in a CaP solution, and such a nanocomposite may thus exhibit multiple physiological activities due to the presence of two or more proteins.

A variety of tissues may be locally cultured on a single substrate by patterning the substrate according to tissue culture form using a technique based on co-precipitating a material containing a protein on a substrate, such as AW glass ceramic. In addition, a substrate may be patterned, for example, by shielding a surface of a substrate with a photosensitive polymer membrane, coating a non-shielded surface of the substrate with a composite containing a first protein, exposing the shielded surface to light to degrade the polymer membrane, shielding the coated surface, forming a coating of a composite containing a second protein, and degrading the polymer membrane of the shielded surface. In addition, the same effects may be achieved by forming a coating of a composite containing a first protein on the surface of a substrate without a patterned shielding membrane, patterning the substrate by mechanical removal of the coating, and forming a coating of a composite containing a second protein on regions other than the patterned regions.

According to the aforementioned process, a single substrate may be locally coated with different biocompatible protein-ceramic composite, and may allow the growth of independent biological tissues even when a biological tissue having a complex structure is required, thereby simplifying implant preparation and implantation.

### INDUSTRIAL APPLICABILITY

As described hereinbefore, the bioactive protein-calcium phosphate ceramic composite promoting the reconstruction of biological tissues according to the present invention exhibits the innate physiological activity of the protein at ambient temperature and under ambient pressure, and regenerates all tissues in the musculoskeletal system regardless of the type, structure or appearance of a base material.

In addition, the present invention provides an effect of imparting multiple physiological activities to a single substrate through a single process, which is based on impregnating two or more proteins selected from among BMP, VEGF, TGF, DBM, and the like in a calcium phosphate solution to yield a protein-calcium phosphate nanocomposite in which two or more proteins are co-precipitated.

1. A bioactive protein-calcium phosphate ceramic composite, prepared by mixing an aqueous solution of calcium phosphate and a protein, and impregnating the calcium phosphate and one or more proteins in a resulting aqueous solution to co-precipitate them on a substrate, wherein the substrate is selected from among metals, ceramics and polymers and is patterned, and different proteins are impregnated in at least two regions among patterned regions.

2. The bioactive protein-calcium phosphate ceramic composite according to claim 1, wherein the aqueous solution of calcium phosphate contains 130˜160 mM NaCl, 1˜3 mM K₂HPO₄.3H₂O and 2˜5 mM CaCl₂, and is adjusted to a pH of 7˜8.

3. The bioactive protein-calcium phosphate ceramic composite according to claim 1, wherein the protein is at least one selected from among bone morphogenetic protein (BMP), VEGF, TGF, and DBM.

4. The bioactive protein-calcium phosphate ceramic composite according to claim 3, wherein a mixture of 20˜40 ml of the aqueous solution of calcium phosphate and 0.1˜100 g/ml of the protein is co-precipitated on the substrate.

5. The bioactive protein-calcium phosphate ceramic composite according to claim 1, wherein the protein layer is 0.1˜1000 μm thick.

6. The bioactive protein-calcium phosphate ceramic composite according to claim 1, wherein the protein-ceramic composite is matured at 20˜30° C. for 1˜5 days after being impregnated.

7. The bioactive protein-calcium phosphate ceramic composite according to claim 1, wherein the ceramic contains calcium and phosphorus within a Ca/P atomic ratio of 1.0˜2.0.

8. A method of preparing a bioactive protein-calcium phosphate ceramic composite, comprising:
forming a pattern on a substrate;
co-precipitating a mixture of an aqueous solution of calcium phosphate and one or more proteins on at least one exposed region of the pattern of the substrate; and
culturing a biological tissue with the co-precipitated proteins.

9. The method according to claim 8, further comprising co-precipitating the mixture of the aqueous solution of calcium phosphate and the proteins on at least one exposed region other than the impregnated regions of the substrate after the co-precipitation step.

10. The method according to claim 8, wherein the aqueous solution of calcium phosphate contains 130˜160 mM NaCl, 1˜3 mM K₂HPO₄.3H₂O and 2˜5 mM CaCl₂, and is adjusted to pH 7˜8.

11. The method according to claim 8, wherein the protein is at least one selected from among bone morphogenetic protein (BMP), VGF), TGF, and DBM.

12. The method according to claim 11, wherein a mixture of 20˜40 ml of the aqueous solution of calcium phosphate and 0.1˜100 g/ml of the protein is co-precipitated on the substrate.

13. The method according to claim 8, wherein the protein layer is 0.1˜1000 μm thick.

14. The method according to claim 8, wherein the protein-ceramic composite is matured at 20˜30° C. for 1˜5 days after being impregnated.

15. The method according to claim 8, wherein the ceramic contains calcium and phosphorus within a Ca/P atomic ratio of 1.0˜2.0.
